(19) United States
(12) Patent Application Publication
(10) Pub. No.: US 2015/0346599 A1
(43) Pub. Date: Dec. 3, 2015

(54) PHOTO-DESTROYABLE QUENCHER AND ASSOCIATED PHOTORESIST COMPOSITION, AND DEVICE-FORMING METHOD

(71) Applicant: Rohm and Haas Electronic Materials LLC, Marlborough, MA (US)

(72) Inventor: Paul J. LaBeaume, Auburn, MA (US)

(21) Appl. No.: 14/289,720

(22) Filed: May 29, 2014

(51) Int. Cl. G03F 7/027; C07D 333/76; G03F 7/30; C07C 381/12

(52) U.S. Cl. CPC G03F 7/027; C07C 381/12; C07D 333/76; G03F 7/30

(57) ABSTRACT

A photo-destroyable quencher has the structure

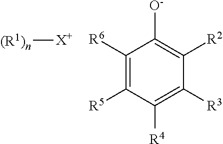

wherein X, n, and R¹-R⁶ are defined herein, and at least one of R², R³, R⁴, R⁵, and R⁶ is halogen, nitro, C₁₋₁₂ fluorinated alkyl, cyano, aldehyde, C₂₋₂₀ ester, C₂₋₂₀ ketone, C₁₋₂₀ sulfoxyl hydrocarbyl, C₁₋₂₀ sulfonyl hydrocarbyl, or sulfonamide. The photo-destroyable quencher exhibits improved solution stability and reduced hygroscopic properties relative to triphenylsulfonium phenolate. A photoresist composition including an acid-sensitive polymer, a photoacid generator, and the photo-destroyable quencher exhibits increased contrast and/or critical dimension uniformity relative to corresponding photoresist compositions comparative photo-destroyable quenchers.

### FIELD

The present invention relates to photo-destroyable quenchers and their use in photoresist compositions.

### INTRODUCTION

Advanced lithographic techniques such as electron beam and extreme ultraviolet lithography have been developed to achieve high quality and smaller feature sizes in microlithography processes, for purposes of forming ever-smaller logic and memory transistors. These advanced lithographic techniques use photoresist compositions, which often include photoacid generators. Photoacid generators generate acid on exposure to incident radiation. In exposed areas of a photoresist, the generated acid reacts with acid-sensitive groups in a photoresist polymer to change the solubility of the polymer, thereby creating a difference in solubility between the exposed and unexposed regions of the photoresist.

Photoresists sometimes include photo-destroyable quenchers in addition to photoacid generators. Like photoacid generators, photo-destroyable quenchers generate acid in exposed areas of a photoresist, but the acid generated by a photo-destroyable quencher is not strong enough to react rapidly with the acid-sensitive groups on the photoresist polymer. (This is what effectively removes the “base” component in the exposed region but leaves an active quencher system in the unexposed area.) However, as the strong acid generated by the photoacid generator in the exposed region migrates to the unexposed region, photo-destroyable quencher in the unexposed region undergoes an anion exchange, losing the anion (conjugate base) of the weak acid, and gaining the anion (conjugate base) of the strong acid. This results in neutralization of strong acid in the unexposed region. So, the concentration of the base is lowered in the exposed region because the photo-destroyable quencher is destroyed in that region. So, the concentration of base is lower in the exposed region than in the unexposed region, which helps the image contrast.

Japanese Patent Application Publication No. JP 2011-154160 A of Shigematsu teaches a photoresist composition comprising triphenylsulfonium phenolate as a photo-destroyable quencher. However, there is a desire for photo-destroyable quenchers that exhibit one or more of increased solution stability, decreased hygroscopic properties, increased lithographic contrast, and increased lithographic critical dimension uniformity.

### SUMMARY

One embodiment is a photo-destroyable quencher having the structure

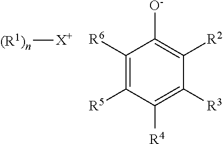

wherein X is iodine or sulfur; n is 2 when X is iodine, and 3 when X is sulfur; each occurrence of R¹ is independently unsubstituted or substituted C₁₋₄₀ hydrocarbyl, or two occurrences of R¹ optionally are bonded to each other to form a ring; and each occurrence of R², R³, R⁴, R⁵, and R⁶ is independently hydrogen, unsubstituted or substituted C₁₋₁₈ hydrocarbyl, halogen, nitro, C₁₋₁₂ fluorinated alkyl, cyano, aldehyde (—C(O)H), C₂₋₂₀ ester (—C(O)OR⁷, wherein R⁷ is C₁₋₁₉ hydrocarbyl), C₂₋₂₀ ketone (—C(O)R⁷, wherein R⁷ is C₁₋₁₉ hydrocarbyl), C₁₋₂₀ sulfonyl hydrocarbyl (—S(O)₂R⁸, wherein R⁸ is C₁₋₂₀ hydrocarbyl), or sulfonamide (—S(O)₂NR⁹₂, wherein each occurrence of R⁹ is independently hydrogen or C₁₋₂₀ hydrocarbyl); provided that at least one of R², R³, R⁴, R⁵, and R⁶ is halogen, nitro, C₁₋₁₂ fluorinated alkyl, cyano, aldehyde (—C(O)H), C₂₋₂₀ ester (—C(O)OR⁷, wherein R⁷ is C₁₋₁₉ hydrocarbyl), C₂₋₂₀ ketone (—C(O)R⁷, wherein R⁷ is C₁₋₁₉ hydrocarbyl), C₁₋₂₀ sulfoxyl hydrocarbyl (—S(O)R⁸, wherein R⁸ is C₁₋₂₀ hydrocarbyl), C₁₋₂₀ sulfonyl hydrocarbyl (—S(O)₂R⁹, wherein R⁹ is C₁₋₂₀ hydrocarbyl), or sulfonamide (—S(O)₂NR¹⁰₂, wherein each occurrence of R¹⁰ is independently hydrogen or C₁₋₂₀ hydrocarbyl); and/or any one or more pairs of adjacent occurrences of R², R³, R⁴, R⁵, and R⁶ are bonded to each other to form an unsubstituted or substituted ring.

Another embodiment is a photoresist composition comprising an acid-sensitive polymer, a photoacid generator, and the above photo-destroyable quencher.

Another embodiment is a coated substrate comprising: (a) a substrate having one or more layers to be patterned on a surface thereof; and (b) a layer of the photoresist composition of over the one or more layers to be patterned.

Another embodiment is a method of forming an electronic device, comprising: (a) applying a layer of a photoresist composition on a substrate; (b) pattern-wise exposing the photoresist composition layer to activating radiation; and (c) developing the exposed photoresist composition layer to provide a resist relief image.

These and other embodiments are described in detail below.

### BRIEF DESCRIPTION OF THE DRAWINGS

FIG. 1 is a chemical scheme for the synthesis of triphenylsulfonium phenolate.

FIG. 2 is a chemical scheme for the synthesis of triphenylsulfonium pentafluorophenolate.

FIG. 3 is a chemical scheme for the synthesis of 5-phenyl-5H-dibenzo[b,d]thiophen-5-ium 2,3,4,5,6-pentafluorophenolate.

FIG. 4 is a chemical scheme for the synthesis of 5-phenyl-5H-dibenzo[b,d]thiophen-5-ium 2,3,5,6-tetrafluorophenolate.

FIG. 5 is a chemical scheme for the synthesis of 5-phenyl-5H-dibenzo[b,d]thiophen-5-ium 3,5-bis(trifluoromethyl)phenolate.

FIG. 6 is a chemical scheme for the synthesis of 5-(4-(tert-butyl)phenyl)-5H-dibenzo[b,d]thiophen-5-ium 2,3,4,5,6-pentafluorophenolate.

FIG. 7 is a chemical scheme for the synthesis of 5-(3,5-dimethyl-4-(2-(((1R,3S,5r,7r)-2-methyladamantant-2-yl)oxy)-2-oxoethoxy)phenyl)-5H-dibenzo[b,d]thiophen-5-ium 2,3,4,5,6-pentafluorophenolate.

FIG. 8 is a chemical scheme for the synthesis of 5-(4-(tert-butyl)phenyl)-5H-dibenzo[b,d]thiophen-5-ium cyclohexylsulfamate.

### DETAILED DESCRIPTION

The present inventor has determined that, relative to triphenylsulfonium phenolate, one or more of increased solution stability, decreased hygroscopic properties (i.e., decreased water absorption), increased lithographic slope, and increased lithographic critical dimension uniformity are provided by a photo-destroyable quencher having a phenolate anion substituted as described herein.

Thus, one embodiment is a photo-destroyable quencher having the structure

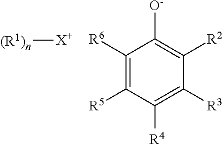

wherein X is iodine or sulfur; n is 2 when X is iodine, and 3 when X is sulfur; each occurrence of R¹ is independently unsubstituted or substituted C₁₋₄₀ hydrocarbyl, or two occurrences of R¹ optionally are bonded to each other to form a ring; and each occurrence of R², R³, R⁴, R⁵, and R⁶ is independently hydrogen, unsubstituted or substituted C₁₋₁₈ hydrocarbyl, halogen, nitro, C₁₋₁₂ fluorinated alkyl, cyano, aldehyde (—C(O)H), C₂₋₂₀ ester (—C(O)OR⁷, wherein R⁷ is C₁₋₁₉ hydrocarbyl), C₂₋₂₀ ketone (—C(O)R⁷, wherein R⁷ is C₁₋₁₉ hydrocarbyl), C₁₋₂₀ sulfonyl hydrocarbyl (—S(O)₂R⁸, wherein R⁸ is C₁₋₂₀ hydrocarbyl), or sulfonamide (—S(O)₂NR⁹₂, wherein each occurrence of R⁹ is independently hydrogen or C₁₋₂₀ hydrocarbyl); provided that at least one of R², R³, R⁴, R⁵, and R⁶ is halogen, nitro, C₁₋₁₂ fluorinated alkyl, cyano, aldehyde (—C(O)H), C₂₋₂₀ ester (—C(O)OR⁷, wherein R⁷ is C₁₋₁₉ hydrocarbyl), C₂₋₂₀ ketone (—C(O)R⁷, wherein R⁷ is C₁₋₁₉ hydrocarbyl), C₁₋₂₀ sulfoxyl hydrocarbyl (—S(O)R⁸, wherein R⁸ is C₁₋₂₀ hydrocarbyl), C₁₋₂₀ sulfonyl hydrocarbyl (—S(O)₂R⁹, wherein R⁹ is C₁₋₂₀ hydrocarbyl), or sulfonamide (—S(O)₂NR¹⁰₂, wherein each occurrence of R¹⁰ is independently hydrogen or C₁₋₂₀ hydrocarbyl); and/or any one or more pairs of adjacent occurrences of R², R³, R⁴, R⁵, and R⁶ are bonded to each other to form an unsubstituted or substituted ring.

Unless otherwise specified, the term “substituted” means including at least one substituent such as a halogen (i.e., F, Cl, Br, I), hydroxyl, amino, thiol, carboxyl, carboxylate, ester (including acrylates, methacrylates, and lactones), amide, nitrile, sulfide, disulfide, nitro, C₁₋₁₈ alkyl (including norbornyl and adamantyl), C₁₋₁₈ alkenyl (including norbornenyl), C₁₋₁₈ alkoxyl, C₂₋₁₈ alkenoxyl (including vinyl ether), C₆₋₁₈ aryl, C₆₋₁₈ aryloxyl, C₇₋₁₈ alkylaryl, or C₇₋₁₈ alkylaryloxyl. “Fluorinated” shall be understood to mean having one or more fluorine atoms incorporated into the group. For example, where a C₁₋₁₈ fluoroalkyl group is indicated, the fluoroalkyl group can include one or more fluorine atoms, for example, a single fluorine atom, two fluorine atoms (e.g., as in a 1,1-difluoroethyl group), three fluorine atoms (e.g., as in a 2,2,2-trifluoroethyl group), or fluorine atoms at each free valence of carbon (e.g., as in a perfluorinated group such as —CF₃, —C₂F₅, —C₃F₇, or —C₄F₉).

Examples of anions in which one or more pairs of adjacent occurrences of R², R³, R⁴, R⁵, and R⁶ are bonded to each other to form an unsubstituted or substituted ring include

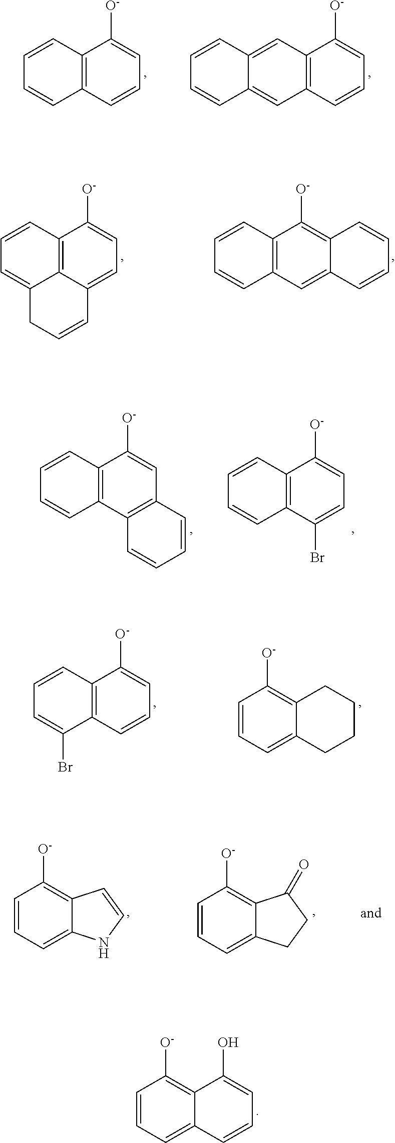

In some embodiments, the phenolate anion of the photo-destroyable quencher has a conjugate acid with a pKₐ of 3 to 9, specifically 5 to 8, more specifically 6 to 8 in aqueous solution at 23° C. pKa values can be measured experimentally or calculated, for example using Advanced Chemistry Development (ACD) Labs Software Version 11.02.

In some embodiments of the photo-destroyable quencher, at least one of R², R³, R⁴, R⁵, and R⁶ is fluorine or C₁₋₁₂ fluorinated alkyl. In some embodiments, at least two of R², R³, R⁴, R⁵, and R⁶ are fluorine or C₁₋₁₂ fluorinated alkyl. In some embodiments, at least three of R², R³, R⁴, R⁵, and R⁶ are fluorine.

In some embodiments, at least one occurrence of R¹ comprises an acid-labile substituent. Acid labile substituents include tertiary esters, acetals, and ketals. Examples of onium ions comprising acid-labile groups include

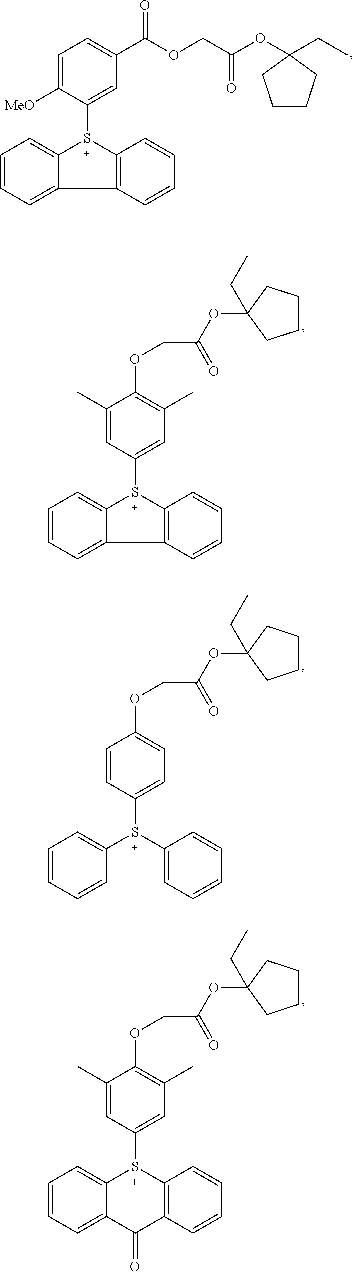

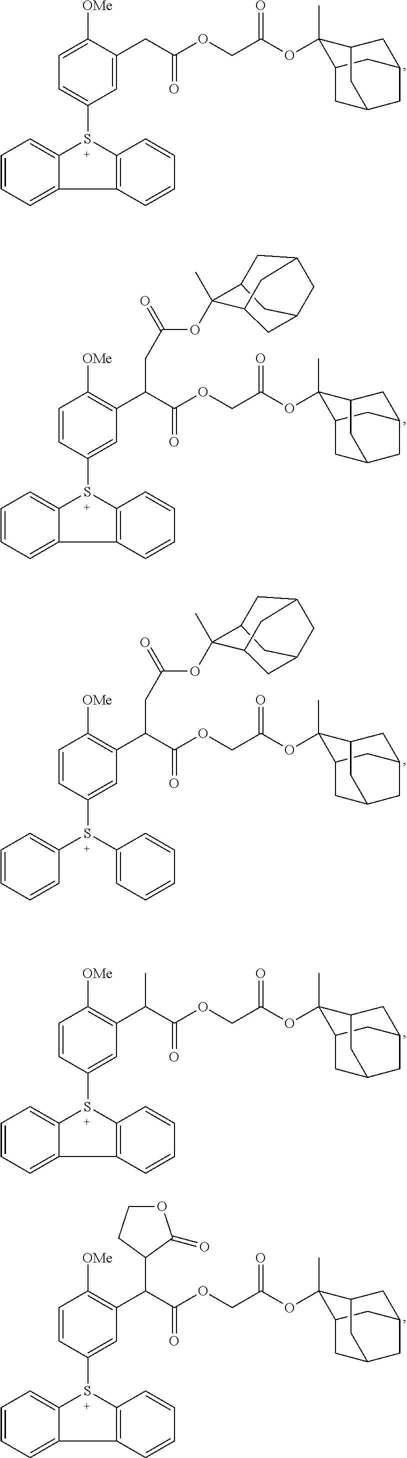

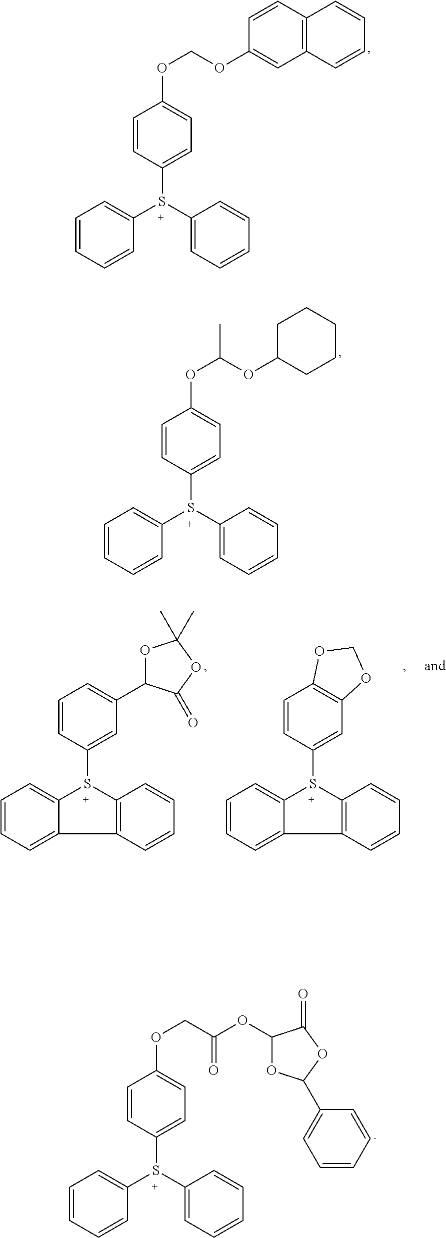

In some embodiments, photo-destroyable quencher has the structure

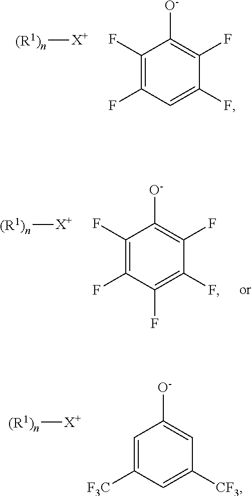

wherein X, n, and R¹ are as defined above.

In some embodiments, the photo-destroyable quencher has the structure

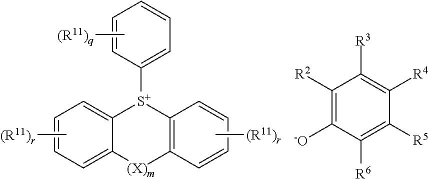

wherein m is 0 (in which case a single bond joins the two adjacent phenyl groups) or 1; q is 0, 1, 2, 3, 4, or 5; each occurrence of r is 0, 1, 2, 3, or 4; R², R³, R⁴, R⁵, and R⁶ are defined as above; each occurrence of R¹¹ is independently unsubstituted or substituted C₁₋₄₀ hydrocarbyl; and X is —O—, —S—, —C(═O)—, —CH₂—, —CH(OH)—, —C(═O)O—, —C(═O)NH—, —C(═O)C(═O)—, —S(═O)—, or —S(═O)₂—.

In some embodiments, the cation of the photo-destroyable quencher has the structure

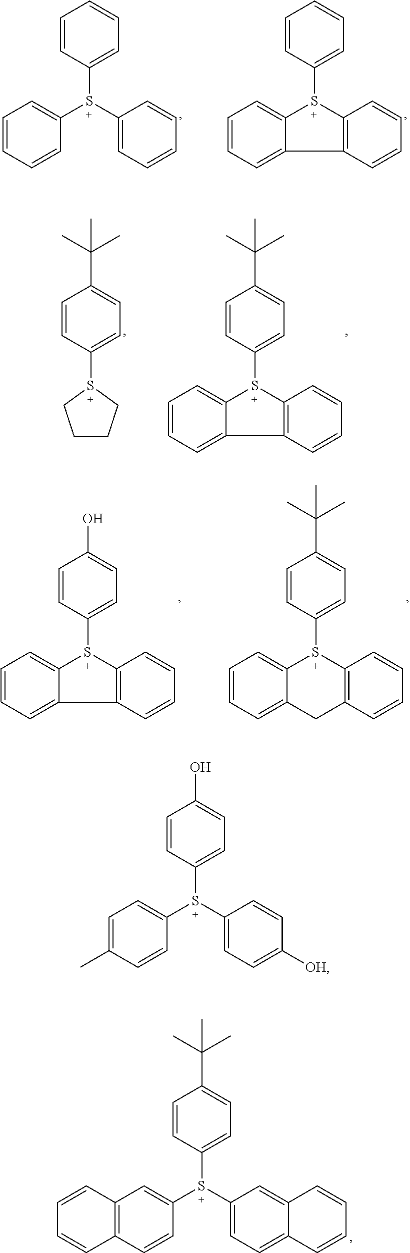

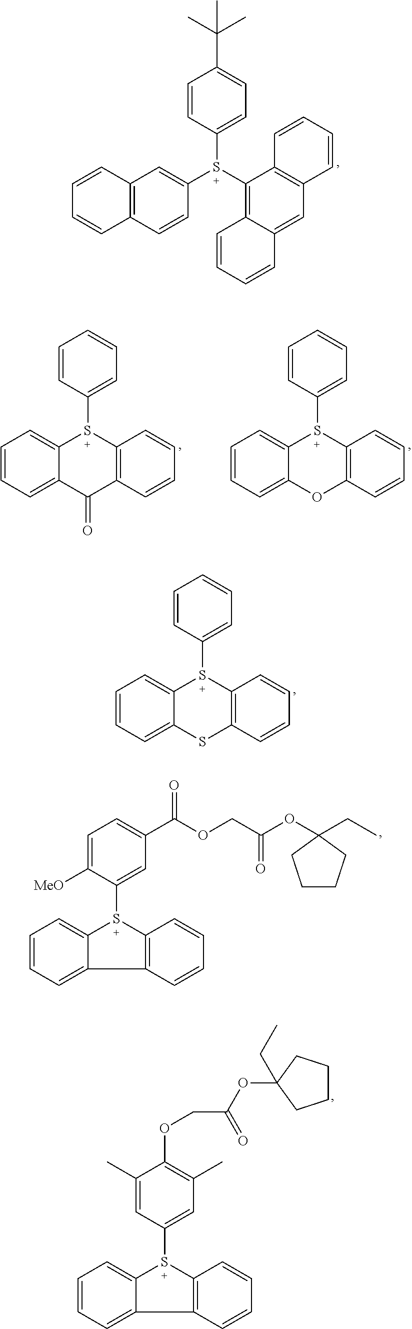

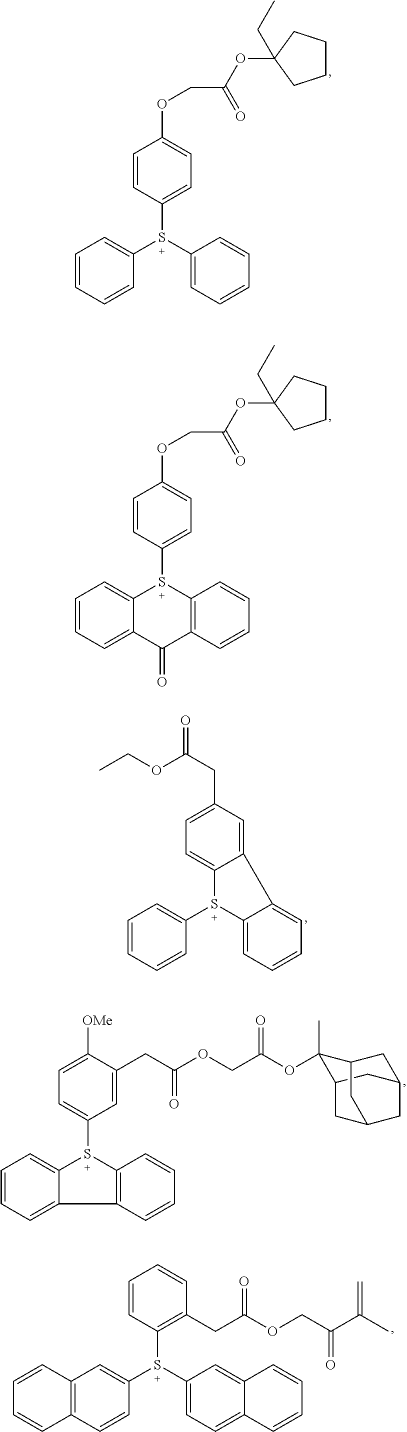

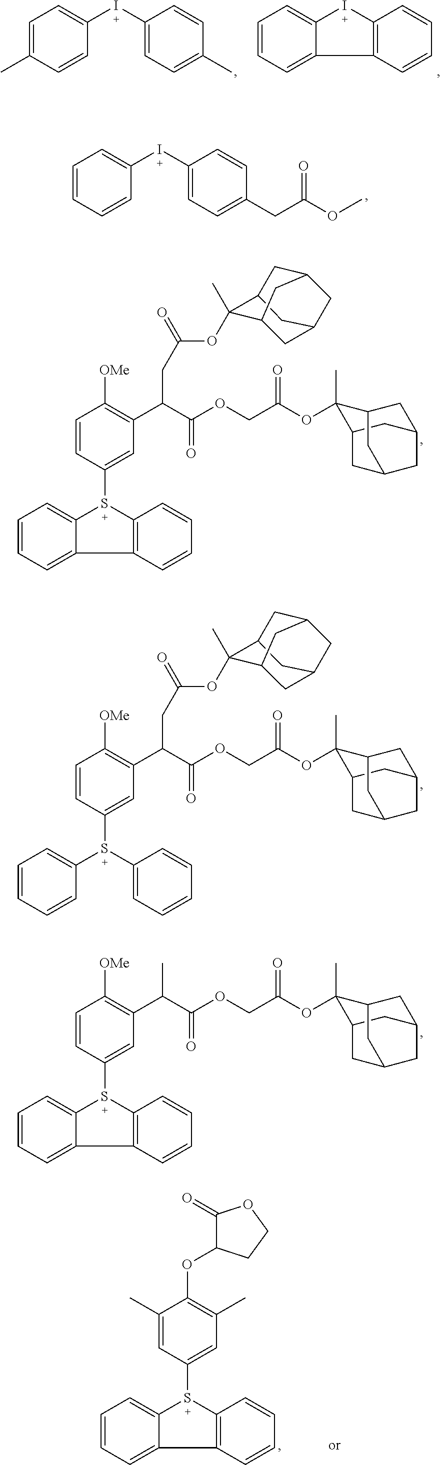

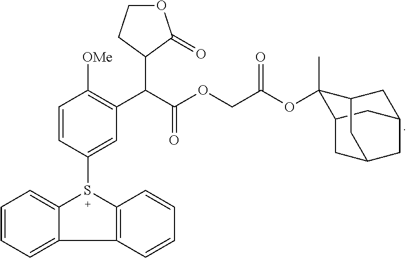

In a very specific embodiment, the photo-destroyable quencher has the structure

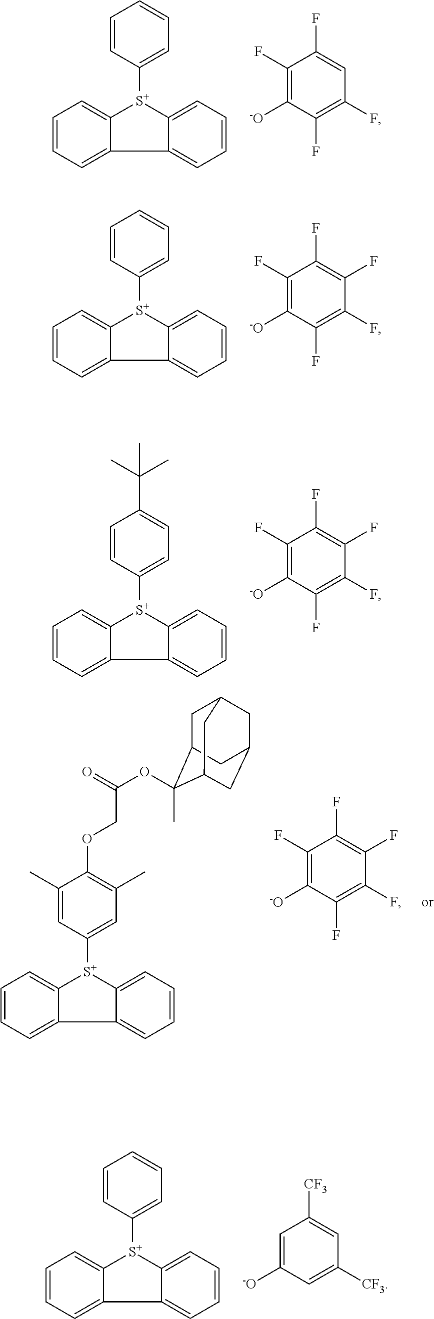

The photo-destroyable quencher is useful as a component of a photoresist composition. Thus, one embodiment is a photoresist composition comprising: an acid-sensitive polymer; a photoacid generator; and a photo-destroyable quencher having the structure

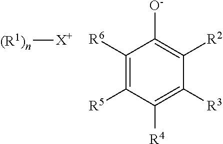

wherein X is iodine or sulfur; n is 2 when X is iodine, and 3 when X is sulfur; each occurrence of R¹ is independently unsubstituted or substituted C₁₋₄₀ hydrocarbyl, or two occurrences of R¹ optionally can be bonded to each other to form a ring; and each occurrence of R², R³, R⁴, R⁵, and R⁶ is independently hydrogen, unsubstituted or substituted C₁₋₁₈ hydrocarbyl, halogen, nitro, C₁₋₁₂ fluorinated alkyl, cyano, aldehyde (—C(O)H), C₂₋₂₀ ester (—C(O)OR⁷, wherein R⁷ is C₁₋₁₉ hydrocarbyl), C₂₋₂₀ ketone (—C(O)R⁷, wherein R⁷ is C₁₋₁₉ hydrocarbyl), C₁₋₂₀ sulfonyl hydrocarbyl (—S(O)₂R⁸, wherein R⁸ is C₁₋₂₀ hydrocarbyl), or sulfonamide (—S(O)₂NR⁹₂, wherein each occurrence of R⁹ is independently hydrogen or C₁₋₂₀ hydrocarbyl); provided that at least one of R², R³, R⁴, R⁵, and R⁶ is halogen, nitro, C₁₋₁₂ fluorinated alkyl, cyano, aldehyde (—C(O)H), C₂₋₂₀ ester (—C(O)OR⁷, wherein R⁷ is C₁₋₁₉ hydrocarbyl), C₂₋₂₀ ketone (—C(O)R⁷, wherein R⁷ is C₁₋₁₉ hydrocarbyl), C₁₋₂₀ sulfoxyl hydrocarbyl (—S(O)R⁸, wherein R⁸ is C₁₋₂₀ hydrocarbyl), C₁₋₂₀ sulfonyl hydrocarbyl (—S(O)₂R⁹, wherein R⁹ is C₁₋₂₀ hydrocarbyl), or sulfonamide (—S(O)₂NR¹⁰₂, wherein each occurrence of R¹⁰ is independently hydrogen or C₁₋₂₀ hydrocarbyl); or any one or more pairs of adjacent occurrences of R², R³, R⁴, R⁵, and R⁶ are bonded to each other to form an unsubstituted or substituted ring.

Acid-sensitive polymers useful for forming a photoresist include the copolymerization products of monomers comprising acid-deprotectable monomers, optionally in combination with one or more of base-soluble monomers, photoacid generating monomers, dissolution rate modifying monomers, and etch-resistant monomers. Any such monomers or combinations of monomers suitable for forming, for example, a 193 nanometer (nm) photoresist polymer can be used. In some embodiments, a combination of monomers is used, which include at least two different monomers selected from a (meth)acrylate monomer having an acid-deprotectable group (deprotection of which yields a base-soluble group), a (meth)acrylate monomer having a lactone functional group, and a (meth)acrylate monomer having a base-soluble group not identical to the acid-deprotectable base soluble group. The acid-sensitive polymer can include at least three different monomers, at least one of which is selected from each of the foregoing monomer types. Other monomers, such as a (meth)acrylate monomer for improving adhesion or etch-resistance, can also be included. The acid-sensitive polymer can incorporate more than one species of at least one monomer type.

Any acid-deprotectable monomer useful for forming a 193 nanometer, extreme ultraviolet, or electron beam photoresist polymer can be used to form the acid-sensitive polymer. These include tertiary alkyl(meth)acrylates, acetal- and ketal-substituted (meth)acrylate esters, and combinations thereof.

Tertiary alkyl(meth)acrylates include, for example,

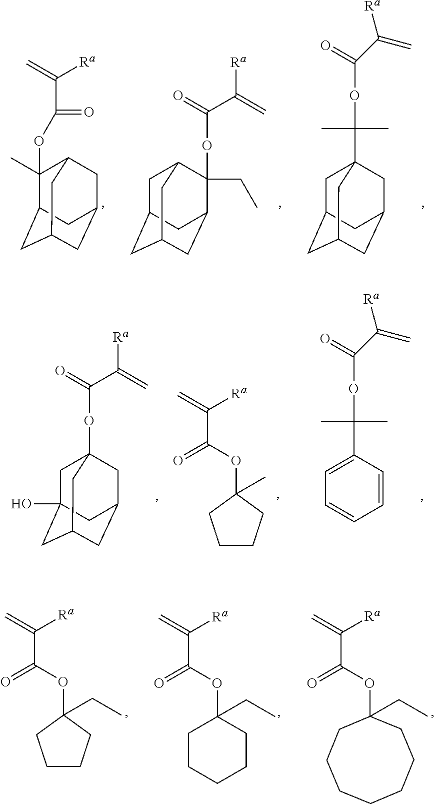

and combinations thereof, wherein R^{a} is H, F, CN, C₁₋₁₀ alkyl, or C₁₋₁₀ fluoroalkyl.

Acetal- and ketal-substituted (meth)acrylate esters include, for example,

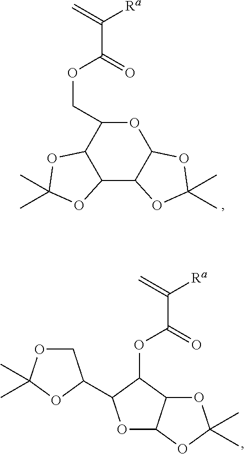

and combinations thereof, wherein R^{a} is H, F, CN, C₁₋₁₀ alkyl, or C₁₋₁₀ fluoroalkyl.

(Meth)acrylate monomers having a lactone functional group include, for example,

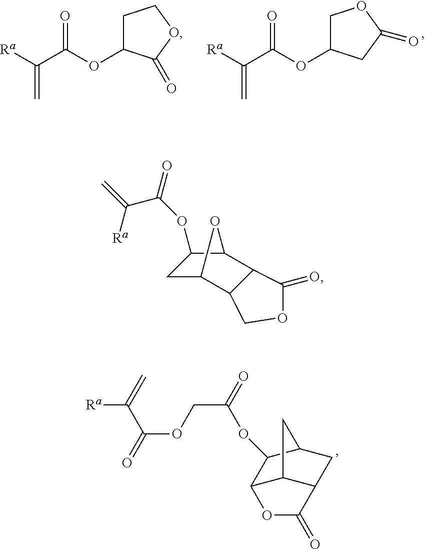

and combinations thereof, wherein R^{a} is H, F, CN, C₁₋₁₀ alkyl, or C₁₋₁₀ fluoroalkyl.

(Meth)acrylate monomer having a base-soluble group include, for example,

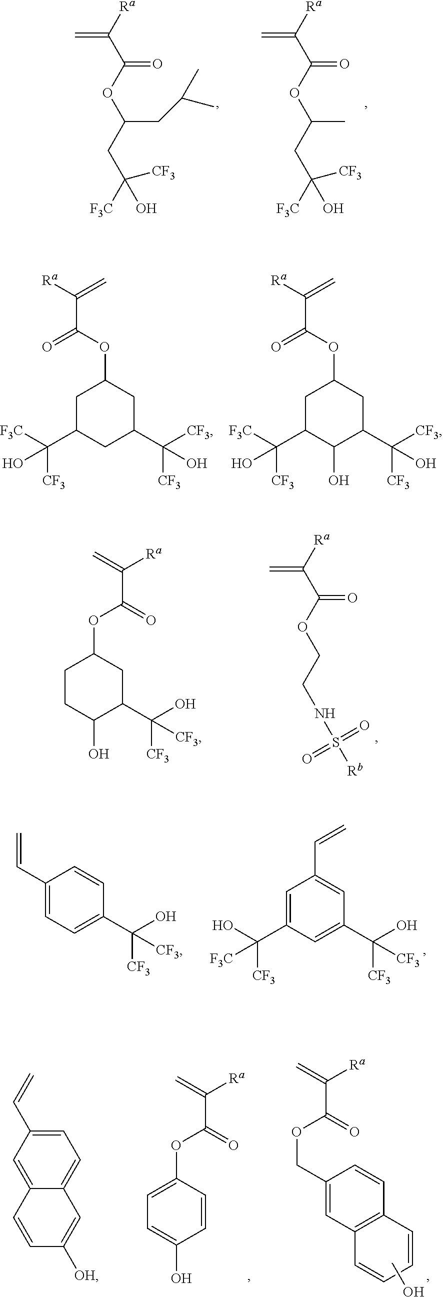

and combinations thereof, wherein R^{a} is H, F, CN, C₁₋₁₀ alkyl, or C₁₋₁₀ fluoroalkyl, and R^{b} is a C₁₋₄ perfluoroalkyl group.

The photoresist composition optionally further includes a second acid-sensitive polymer, a second photoacid generator compound, a second photo-destroyable quencher, an amine or amide additive to adjust photospeed and/or acid diffusion, a solvent, a surfactant, or a combination thereof.

The photoresist composition can include an amine or amide compound. These compounds are sometimes referred to as “quenchers” but are chemically distinct from the photo-destroyable quencher. The amine or amide compounds include C₁₋₃₀ organic amines, imines, or amides, or can be a C₁₋₃₀ quaternary ammonium salt of a strong base (e.g., a hydroxide or alkoxide) or a weak base (e.g., a carboxylate). Exemplary amine or amide compounds include amines such as Troger's base, hindered amines such as diazabicycloundecene (DBU) and diazabicyclononene (DBN), N-protected amines such as N-t-butylcarbonyl-1,1-bis(hydroxymethyl)-2-hydroxyethylamine, and ionic compounds including quaternary alkyl ammonium salts such as tetrabutylammonium hydroxide (TBAH) and tetrabutyl ammonium lactate.

Examples of second photo-destroyable quenchers include triphenylsulfonium hydroxide, triphenylsulfonium 3-hydroxyadamantane carboxylate, triphenylsulfonium camphorsulfonate, and t-butylphenyldibenzothiophenium 1-adamantanecarboxylate.

Solvents generally suitable for dissolving, dispensing, and coating the components include anisole, alcohols including ethyl lactate, methyl 2-hydroxybutyrate (HBM), 1-methoxy-2-propanol (also referred to as propylene glycol methyl ether, PGME), and 1-ethoxy-2 propanol, esters including n-butyl acetate, 1-methoxy-2-propyl acetate (also referred to as propylene glycol methyl ether acetate, PGMEA), methoxyethyl propionate, ethoxyethyl propionate, and gamma-butyrolactone, ketones including cyclohexanone and 2-heptanone, and combinations thereof.

Surfactants include fluorinated and non-fluorinated surfactants, and are preferably non-ionic. Exemplary fluorinated non-ionic surfactants include perfluoro C₄ surfactants such as FC-4430 and FC-4432 surfactants, available from 3M Corporation; and fluorodiols such as POLYFOX™ PF-636, PF-6320, PF-656, and PF-6520 fluorosurfactants from Omnova.

The acid-sensitive polymer can be present in the photoresist composition in an amount of 50 to 99 weight percent, specifically 55 to 95 weight percent, more specifically 60 to 90 weight percent, and still more specifically 65 to 90 based on the total weight of solids in the photoresist composition. It will be understood that “polymer” used in this context of a component in a photoresist can mean only the acid-sensitive polymer described herein, or a combination of the acid-sensitive polymer with another polymer useful in a photoresist. The photoacid generator can be present in the photoresist composition in an amount of 0.01 to 40 weight percent, specifically 0.1 to 20 weight percent, based on the total weight of solids in the photoresist composition. Where a polymer-bound photoacid generator is used, the polymer-bound photoacid generator as the corresponding monomer is present in the same amount. In some embodiments, photoresist composition comprises polymer-bound photoacid generator and a photoacid generator additive. The photo-destroyable quencher can be present in the photoresist composition in an amount of 0.01 to 20 weight percent, specifically 0.1 to 10 weight percent, more specifically 0.5 to 3 weight percent, based on the total weight of solids in the photoresist composition. A surfactant can be included in the photoresist composition in an amount of 0.01 to 5 weight percent, specifically 0.1 to 4 weight percent, and still more specifically 0.2 to 3 weight percent, based on the total weight of solids in the photoresist composition. Other additives such as embedded barrier layer (EBL) materials for immersion lithography applications can be included in amounts of less than or equal to 30 weight percent, specifically less than or equal to 20 weight percent, or more specifically less than or equal to 10 weight percent, based on the total weight of solids. The total solids content of the photoresist composition can be 0.5 to 50 weight percent, specifically 1 to 45 weight percent, more specifically 2 to 40 weight percent, and still more specifically 5 to 35 weight percent, based on the total weight of solids and solvent. It will be understood that the “solids” includes acid-sensitive polymer, photoacid generator, photo-destroyable quencher, surfactant, and any optional additives, exclusive of solvent.

The photoresist composition can be used to form a film comprising the photoresist, where the film on the substrate constitutes a coated substrate. Such a coated substrate includes: (a) a substrate having one or more layers to be patterned on a surface thereof; and (b) a layer of the photoresist composition over the one or more layers to be patterned. Preferably, patterning is carried out using ultraviolet radiation at wavelength of less than 248 nm, and in particular, at 193 nm or 13.4 nm. A method of forming an electronic device includes: (a) applying a layer of the photoresist composition on a substrate; (b) pattern-wise exposing the photoresist composition layer to activating radiation; and (c) developing the exposed photoresist composition layer to provide a resist relief image. In some embodiments, the radiation is extreme ultraviolet (EUV) or electron beam (e-beam) radiation.

Developing the pattern can be accomplished by either positive tone development (PTD) in which the pattern-wise exposed region is removed by the action of an aqueous base developer such as aqueous tetramethylammonium hydroxide (TMAH). An exemplary positive tone developer is 0.26 Normal aqueous TMAH. Alternatively, the same pattern-wise exposure can be developed using an organic solvent developer to provide a negative tone development (NTD) in which the unexposed region of a pattern is removed by the action of a negative tone developer. Useful solvents for negative tone development include those also useful for dissolving, dispensing, and coating. Exemplary negative tone developer solvents include propylene glycol methyl ether acetate (PGMEA), methyl 2-hydroxyisobutyrate (HBM), methoxyethyl propionate, ethoxyethyl propionate, and gamma-butyrolactone, cyclohexanone, 2-heptanone, and combinations thereof. A method of making a pattern thus includes pattern-wise exposing a photoresist composition layer with actinic radiation, and developing the pattern by treatment with an aqueous alkaline developer to form a positive tone relief image, or with an organic solvent developer to form a negative tone relief image.

Substrates can be any dimension and shape, and are preferably those useful for photolithography, such as silicon, silicon dioxide, silicon-on-insulator (SOI), strained silicon, gallium arsenide, coated substrates including those coated with silicon nitride, silicon oxynitride, titanium nitride, tantalum nitride, ultrathin gate oxides such as hafnium oxide, metal or metal coated substrates including those coated with titanium, tantalum, copper, aluminum, tungsten, alloys thereof, and combinations thereof. The surfaces of substrates herein can include critical dimension layers to be patterned including, for example, one or more gate-level layers or other critical dimension layer on the substrates for semiconductor manufacture. The substrates can be formed as circular wafers having dimensions such as, for example, 200 millimeters, 300 millimeters, or larger in diameter, or other dimensions useful for wafer fabrication.

The invention is further illustrated by the following non-limiting examples.

### Comparative Example 1

Triphenylsulfonium phenolate.

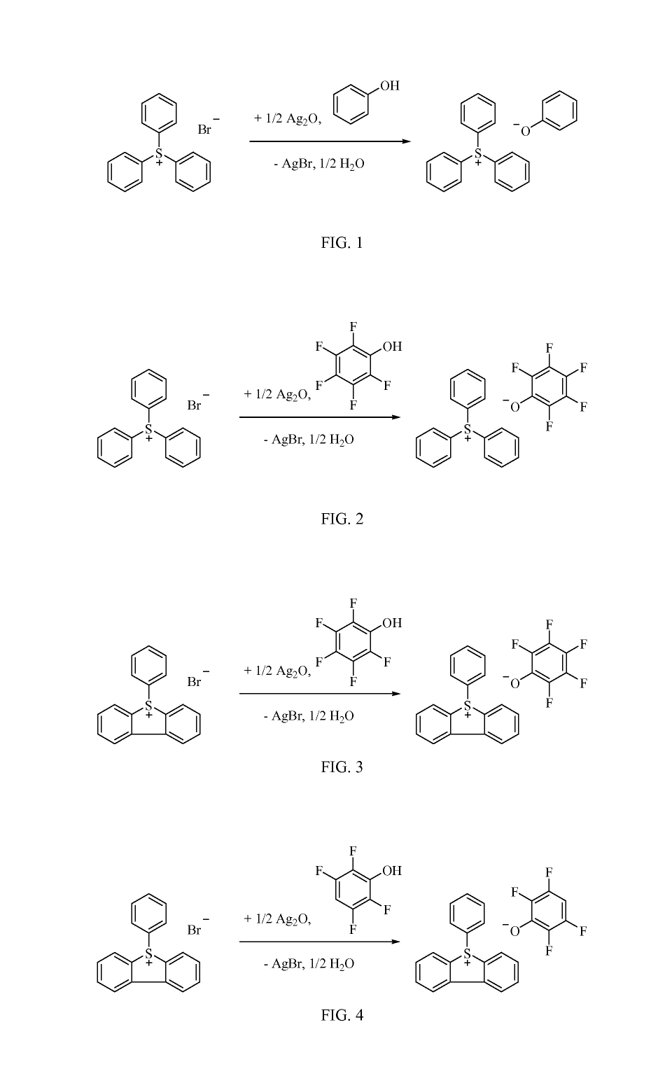

The reaction is summarized in FIG. 1. Silver oxide (2.84 grams, 12.2 millimoles) was added to a solution of triphenylsulfonium bromide (4.00 grams, 11.6 millimoles) in methanol (50 milliliters) and stirred at room temperature for 4 hours. The mixture was filtered through CELITE™, which was washed with methanol (50 milliliters), and phenol (1.10 grams, 11.6 millimoles) was added to the combined organic layers and stirred at room temperature for 2 hours. The solution was concentrated to a viscous oil and added to methyl t-butyl ether (MTBE):heptanes (1:1 volume/volume, 300 milliliters) and vigorously stirred for 30 minutes. The organic layer was decanted from the resulting precipitate, MTBE (250 milliliters) was added, and the resulting suspension was vigorously stirred. The precipitate was filtered and washed with MTBE (2×150 milliliters) to afford triphenylsulfonium phenolate (4.14 grams, 99%) as a white hygroscopic solid. ¹H NMR (300 MHz, (CD₃)₂SO) δ: 7.75-7.89 (m, 15H), 6.75 (t, J=7.2 Hz, 2H), 6.22 (d, J=7.1 Hz, 2H), 5.97 (t, J=7.2 Hz, 1H).

### Example 1

### Triphenylsulfonium pentafluorophenolate

The reaction is summarized in FIG. 2. Silver oxide (2.84 grams, 12.2 millimoles) was added to a solution of triphenylsulfonium bromide (4.00 grams, 11.6 millimoles) in methanol (50 milliliters) and stirred at room temperature for 4 hours. The mixture was filtered through CELITE™, which was washed with methanol (50 milliliters), and pentafluorophenol (2.13 grams, 11.6 millimoles) was added to the combined organic layers and stirred at room temperature for 2 hours. The solution was concentrated to a viscous oil and added to MTBE:heptanes (1:1, 300 milliliters) and vigorously stirred for 30 minutes. The organic layer was decanted from the precipitated oil and MTBE added (250 milliliters) and vigorously stirred. The resulting precipitate was filtered and washed with MTBE (2×150 milliliters) to afford triphenylsulfonium pentafluorophenolate (3.35 grams, 65%) as a white hygroscopic solid. ¹H NMR (300 MHz, (CD₃)₂SO) δ: 7.67-7.94 (m, 15H). ¹⁹F NMR (300 MHz, (CD₃)₂SO) δ: −172.53 (m, 4F), −196.79 (m, 1F).

### Example 2

### 5-Phenyl-5H-dibenzo[b,d]thiophen-5-ium 2,3,4,5,6-pentafluorophenolate

The reaction is summarized in FIG. 3. Silver oxide (3.57 grams, 15.4 millimoles) was added to a solution of 5H-dibenzo[b,d]thiophen-5-ium bromide (5.00 grams, 14.7 millimoles) in methanol (50 milliliters) and stirred overnight. The reaction mixture was filtered through CELITE™, which was washed with methanol (50 milliliters) and the organic layers combined. Tetrafluorophenol (2.70 grams, 14.7 millimoles) was then added and stirred at room temperature for 4 hours. The solution was concentrated to an oil which was precipitated into MTBE (250 milliliters). The solid was filtered and washed with MTBE (2×200 milliliters) to afford 5-phenyl-5H-dibenzo[b,d]thiophen-5-ium 2,3,4,5,6-pentafluorophenolate (4.95 grams, 76%) as a white solid. ¹H NMR (300 MHz, (CD₃)₂SO) δ: 8.54 (d, J=7.5 Hz, 2H), 8.42 (d, J=7.8 Hz, 2H), 7.97 (t, J=7.5 Hz, 2H), 7.77 (t, J=7.5 Hz, 2H), 7.52-7.77 (m, 5H). ¹⁹F NMR (300 MHz, (CD₃)₂SO) δ: −172.45 (m, 4F), −196.83 (m, 1F).

### Example 3

### 5-Phenyl-5H-dibenzo[b,d]thiophen-5-ium 2,3,5,6-tetrafluorophenolate

The reaction is summarized in FIG. 4. Silver oxide (3.57 grams, 15.4 millimoles) was added to a solution of 5H-dibenzo[b,d]thiophen-5-ium bromide (5.00 grams, 14.7 millimoles) in methanol (50 milliliters) and stirred overnight. The reaction mixture was filtered through CELITE™, which was washed with methanol (50 milliliters) and the organic layers combined. 2,3,5,6-tetrafluorophenol (2.42 grams, 14.7 millimoles) was then added and stirred at room temperature for 4 hours. The solution was concentrated to an oil which was precipitated into MTBE (250 milliliters). The solid was filtered and washed with MTBE (2×200 milliliters) to afford 5-phenyl-5H-dibenzo[b,d]thiophen-5-ium 2,3,5,6-tetrafluorophenolate (3.00 grams, 48%) as a white solid. ¹H NMR (300 MHz, (CD₃)₂SO) δ: 8.54 (d, J=7.5 Hz, 2H), 8.43 (d, J=7.5 Hz, 2H), 7.96 (t, J=7.8 Hz, 2H), 7.76 (t, J=7.8 Hz, 2H), 7.54-7.74 (m, 5H), 5.59-5.73 (m, 1H). ¹⁹F NMR (300 MHz, (CD₃)₂SO) δ: −148.02 (m, 2F), −169.85 (m, 2F).

### Example 4

### 5-Phenyl-5H-dibenzo[b,d]thiophen-5-ium 3,5-bis(trifluoromethyl)-phenolate

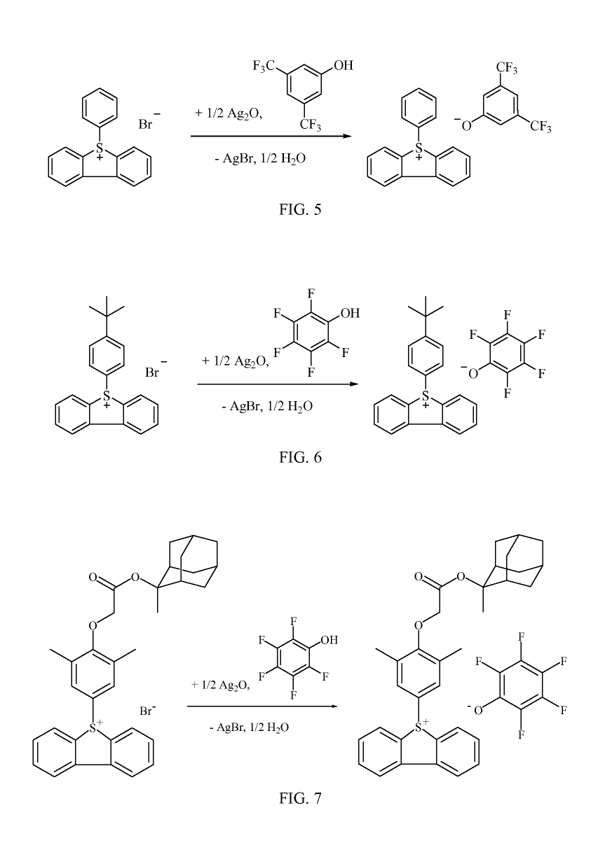

The reaction is summarized in FIG. 5. Silver oxide (3.57 grams, 15.4 millimoles) was added to a solution of 5H-dibenzo[b,d]thiophen-5-ium bromide (5.00 grams, 14.7 millimoles) in methanol (50 milliliters) and stirred overnight. The reaction mixture was filtered through CELITE™, which was washed with methanol (50 milliliters) and the organic layers combined. 3,5-bis(trifluoromethyl)phenol (3.37 grams, 14.7 millimoles) was then added and stirred at room temperature for 4 hours. The solution was concentrated to an oil which was suspended in MTBE:heptanes (1:1, 250 milliliters) and vigorously stirred overnight. The solid was filtered and washed with MTBE (2×200 milliliters) to afford 5-phenyl-5H-dibenzo[b,d]thiophen-5-ium 3,5-bis(trifluoromethyl)-phenolate (3.05 grams, 42%) as a white solid. ¹H NMR (300 MHz, (CD₃)₂SO) δ: 8.53 (d, J=7.8 Hz, 2H), 8.41 (d, J=7.8 Hz, 2H), 7.96 (t, J=7.8 Hz, 2H), 7.76 (t, J=8.1 Hz, 2H), 7.52-7.72 (m, 5H), 6.32-6.37 (vis brs, 2H), 6.15-6.20 (vis brs, 1H). ¹⁹F NMR (300 MHz, (CD₃)₂SO) δ: −61.93 (s, 6F).

### Example 5

### 5-(4-(tert-Butyl)phenyl)-5H-dibenzo[b,d]thiophen-5-ium 2,3,4,5,6-pentafluorophenolate

The reaction is summarized in FIG. 6. Silver oxide (2.45 grams, 10.6 millimoles) was added to a solution of 5-(4-(tert-butyl)phenyl)-5H-dibenzo[b,d]thiophen-5-ium bromide (4.00 grams, 10.1 millimoles) in methanol (50 milliliters) and stirred overnight. The reaction mixture was filtered through CELITE™, which was washed with methanol (50 milliliters) and the organic layers combined. Tetrafluorophenol (1.85 grams, 10.1 millimoles) was then added and stirred at room temperature for 4 hours. The solution was concentrated to an oil which was precipitated into MTBE:heptanes (1:1, 250 milliliters). The solid was filtered and washed with MTBE (2×200 milliliters) to afford 5-(4-(tert-butyl)phenyl)-5H-dibenzo[b,d]thiophen-5-ium 2,3,4,5,6-pentafluorophenolate (4.66 grams, 92%) as a white solid. ¹H NMR (300 MHz, (CD₃)₂SO) δ: 8.53 (d, J=7.8 Hz, 2h), 8.38 (d, J=7.8 Hz, 2H), 7.96 (t, J=8.1 Hz, 2H), 7.76 (t, J=7.8 Hz, 2H), 7.61 (d, J=8.7 Hz, 2H), 7.52 (d, J=8.7 Hz, 2H), 1.23 (s, 9H). ¹⁹F NMR (300 MHz, (CD₃)₂SO) δ: −169.16 (m, 2F), −170.93 (m, 2F), −189.87 (m, 1F).

### Example 6

### 5-(3,5-Dimethyl-4-(2-(((1R,3S,5r,7r)-2-methyladamantant-2-yl)oxy)-2-oxoethoxy)phenyl)-5H-dibenzo[b,d]thiophen-5-ium 2,3,4,5,6-pentafluorophenolate

The reaction is summarized in FIG. 7. Silver oxide (2.22 grams, 9.60 millimoles) was added to a solution of 5-(3,5-dimethyl-4-(2-(((1R,3S,5r,7r)-2-methyladamantant-2-yl)oxy)-2-oxoethoxy)phenyl)-5H-dibenzo[b,d]thiophen-5-ium chloride (5.00 grams, 9.14 millimoles) and pentafluorophenol (1.77 grams, 9.60 millimoles) in methanol (100 milliliters) and stirred at room temperature for 4 hours. The reaction mixture was filtered through CELITE™, which was washed with methanol (100 milliliters), the organic layers combined and concentrated to a viscous oil which was dissolved in minimal acetone which was then fully dissolved in MTBE (250 milliliters) and vigorously stirred overnight. The resulting brown solids were discarded and the mother liquor concentrated to about 20 milliliters which was precipitated into MTBE:heptanes (2:3, 250 milliliters) as an oil. The solution was decanted, the oil was washed with MTBE:heptanes (2:3, 2×100 milliliters), redissolved in acetone and concentrated to dryness to afford 5-(3,5-dimethyl-4-(2-(((1R,3S,5r,7r)-2-methyladamantant-2-yl)oxy)-2-oxoethoxy)phenyl)-5H-dibenzo[b,d]thiophen-5-ium 2,3,4,5,6-pentafluorophenolate (3.00 grams, 47%) as a white solid. ¹H NMR (300 MHz, (CD₃)₂CO) δ: 8.51 (d, J=8.1 Hz, 2H), 8.49 (d, J=8.1 Hz, 2H), 8.00 (dt, J=8.1, 0.9 Hz, 2H), 7.79 (dt, J=8.1, 0.9 Hz, 2H), 7.48 (s, 2H), 4.56 (s, 2H), 1.63 (s, 3H), 1.50-2.09 (m, 14H). ¹⁹F NMR (300 MHz, (CD₃)₂CO) δ: −169.81 (m, 2F), −172.88 (m, 3F).

### Comparative Example 2

### 5-(4-(tert-Butyl)phenyl)-5H-dibenzo[b,d]thiophen-5-ium cyclohexylsulfamate

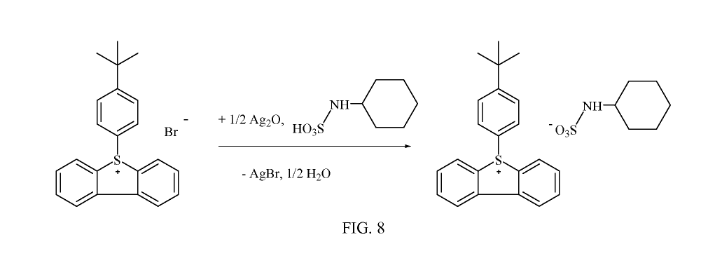

The reaction is summarized in FIG. 8. Silver oxide (7.35 grams, 31.7 millimoles) was added to a solution of 5-(4-(tert-butyl)phenyl)-5H-dibenzo[b,d]thiophen-5-ium bromide (12.00 grams, 30.2 millimoles) in methanol (150 milliliters) and stirred overnight. The reaction mixture was filtered through CELITE™, which was washed with methanol (100 milliliters) and the organic layers combined. Cyclamic acid (5.41 grams, 30.2 millimoles) was then added and stirred at room temperature overnight. The solution was concentrated to an oil which was dissolved in minimal dichloromethane and precipitated into MTBE (500 milliliters). The crude solid was filtered, suspended in MTBE:tetrahydrofuran (2:1, 300 milliliters), heated to 40° C. for 1 hour, cooled to room temperature, filtered and washed with MTBE:tetrahydrofuran (2:1, 300 milliliters) to afford 5-(4-(tert-butyl)phenyl)-5H-dibenzo[b,d]thiophen-5-ium cyclohexylsulfamate (11.9 grams, 80%) as a white solid. ¹H NMR (300 MHz, (CD₃)₂SO) δ: 8.54 (d, J=7.8 Hz, 2H), 8.38 (d, J=8.1 Hz, 2H), 7.90 (t, J=7.5 Hz, 2H), 7.76 (t, J=7.5 Hz, 2H), 7.63 (d, J=7.8 Hz, 2H), 7.53 (d, J=7.8 Hz, 2H), 4.20-5.50 (brs, NH), 3.57-3.65 (m, 1H), 2.84-2.95 (m, 1H), 1.85-1.98 (m, 2H), 1.72-1.82 (m, 1H), 1.55-1.86 (m, 2H), 1.42-1.53 (m, 1H), 1.24 (2, 9H), 1.00-1.35 (m, 3H).

### Preparative Example 1

This example describes the preparation of polymer with acid generator units derived from 5-(4-(2-(1-ethylcyclopentyloxy)-2-oxoethoxy)-3,5-dimethylphenyl)-5H-dibenzo[b,d]thiophenium 1,1-difluoro-2-(methacryloyloxy)ethanesulfonate. A heel solution was made by dissolving 2-phenylpropan-2-yl methacrylate (0.39 gram), 2-oxotetrahydrofuran-3-yl methacrylate (0.33 gram), 3,5-bis(1,1,1,3,3,3-hexafluoro-2-hydroxypropan-2-yl)cyclohexyl methacrylate (0.57 gram), and 5-(4-(2-(1-ethylcyclopentyloxy)-2-oxoethoxy)-3,5-dimethylphenyl)-5H-dibenzo[b,d]thiophenium 1,1-difluoro-2-(methacryloyloxy)ethanesulfonate (0.31 gram) in 12.81 grams acetonitrile/tetrahydrofuran (2:1 volume/volume). A feed solution was prepared by dissolving 2-phenylpropan-2-yl methacrylate (185.54 grams, 0.967 moles), 2-oxotetrahydrofuran-3-yl methacrylate (204.27 grams, 1.26 moles), 3,5-bis(1,1,1,3,3,3-hexafluoro-2-hydroxypropan-2-yl)cyclohexyl methacrylate (127.98 grams, 0.29 mole), and 5-(4-(2-(1-ethylcyclopentyloxy)-2-oxoethoxy)-3,5-dimethylphenyl)-5H-dibenzo[b,d]thiophenium 1,1-difluoro-2-(methacryloyloxy)ethanesulfonate (81.5 grams, 0.132 mole) in 606 grams ethyl lactate/γ-butyrolactone (30:70 volume/volume). An initiator solution was prepared by dissolving 65.96 grams initiator (obtained as Wako V-65) in 66 grams acetonitrile/tetrahydrofuran (2:1 volume/volume). The polymerization was carried out in a 2 liter 3-neck roundbottom flask fitted with a water condenser and a thermometer to monitor the reaction in the flask. The contents were stirred using an overhead stirrer. The reactor was charged with the heel solution and the contents were heated to 75° C. The feed solution and the initiator solution were fed into the reactor using syringe pumps over a 4 hour period. The contents were then stirred for additional 2 hours, after which the reaction was quenched using hydroquinone (2.0 grams). The contents were cooled to room temperature and precipitated twice out of 10-fold (by weight) diisopropyl ether/methanol 95:5 (weight/weight). After each precipitation step, the polymer obtained was dried under vacuum at 50° C. for 24 hours to yield 500 grams polymer.

### Preparative Example 2

This example describes the preparation of polymer with acid generator units derived from 5-phenyl-5H-dibenzo[b,d]thiophenium 1,1-difluoro-2-(methacryloyloxy)ethanesulfonate (PDBT-F2). The procedure of Preparative Example 1 was used, except that 5-phenyl-5H-dibenzo[b,d]thiophenium 1,1-difluoro-2-(methacryloyloxy)ethanesulfonate was used in place of 5-(4-(2-(1-ethylcyclopentyloxy)-2-oxoethoxy)-3,5-dimethylphenyl)-5H-dibenzo[b,d]thiophenium 1,1-difluoro-2-(methacryloyloxy)ethanesulfonate.

### Preparative Example 3

This example describes the preparation of polymer with acid generator units derived from 5-(4-(tert-butyl)phenyl)-5H-dibenzo[b,d]thiophen-5-ium 1,1-difluoro-2-(methacryloyloxy)ethanesulfonate (TBPDBT-F2). The procedure of Preparative Example 1 was used, except that 5-(4-(tert-butyl)phenyl)-5H-dibenzo[b,d]thiophen-5-ium 1,1-difluoro-2-(methacryloyloxy)ethanesulfonate was used in place of 5-(4-(2-(1-ethylcyclopentyloxy)-2-oxoethoxy)-3,5-dimethylphenyl)-5H-dibenzo[b,d]thiophenium 1,1-difluoro-2-(methacryloyloxy)ethanesulfonate.

### Example 7

This example describes the preparation of a photoresist composition containing the inventive Example 5 photo-destroyable quencher. A positive-tone photoresist composition was prepared by combining 7.907 grams of a 10 weight percent solution of the Preparative Example 3 polymer in ethyl lactate; 9.794 grams of a 2 weight percent solution of the acid generator 5-(4-(2-(1-methyladamantyl)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in ethyl lactate; 0.474 gram of a 0.5 weight percent solution of tetrakis(2-hydroxypropy)ethylenediamine in ethyl lactate; 0.515 gram of a 2 weight percent solution of the Example 5 photo-destroyable quencher in ethyl lactate; 0.158 gram of a 0.5 weight percent solution of fluorinated surfactant (Omnova PolyFox™ PF-656) in ethyl lactate; 9.452 grams of ethyl lactate; and 11.70 grams of 2-hydroxyisobutyric acid methyl ester. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to extreme ultraviolet (EUV) radiation. The imaged resist layer was then baked at 100° C. for 60 seconds and then developed with an aqueous alkaline composition.

### Comparative Example 3

A positive-tone photoresist composition was prepared by combining 15.815 grams of a 10 weight percent solution of the Preparative Example 3 polymer in ethyl lactate; 19.590 grams of a 2 weight percent solution of the acid generator 5-(4-(2-(1-methyladamantyl)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in ethyl lactate; 0.949 gram of a 0.5 weight percent solution of tetrakis(2-hydroxypropyl)ethylenediamine in ethyl lactate; 0.515 gram of a 2 weight percent solution of the Comparative Example 2 photo-destroyable quencher in ethyl lactate; 0.316 gram of a 0.5 weight percent solution of fluorinated surfactant (Omnova PolyFox™ PF-656) in ethyl lactate; 18.909 grams of ethyl lactate; and 23.400 grams of 2-hydroxyisobutyric acid methyl ester. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

### Comparative Example 4

A positive-tone photoresist composition was prepared by combining 9.881 grams of a 10 weight percent solution of the Preparative Example 3 polymer in ethyl lactate; 12.240 grams of a 2 weight percent solution of the acid generator 5-(4-(2-(1-methyladamantyl)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in ethyl lactate; 0.593 gram of a 0.5 weight percent solution of tetrakis(2-hydroxypropyl)ethylenediamine in ethyl lactate; 0.515 gram of a 2 weight percent solution of the photo-destroyable quencher 5-(4-(tert-butyl)phenyl)-5H-dibenzo[b,d]thiophen-5-ium (1r,3s,5R,7S)-3-hydroxyadamantane-1-carboxylate in ethyl lactate; 0.198 gram of a 0.5 weight percent solution of fluorinated surfactant (Omnova PolyFox™ PF-656) in ethyl lactate; 11.805 grams of ethyl lactate; and 14.625 grams of 2-hydroxyisobutyric acid methyl ester. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

### Comparative Example 5

A positive-tone photoresist composition was prepared by combining 9.885 grams of a 10 weight percent solution of the Preparative Example 3 polymer in ethyl lactate; 12.245 grams of a 2 weight percent solution of the acid generator 5-(4-(2-(1-methyladamantyl)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in ethyl lactate; 0.634 gram of a 0.5 weight percent solution of tetrakis(2-hydroxypropyl)ethylenediamine in ethyl lactate; 0.593 gram of a 2 weight percent solution of the photo-destroyable quencher 5-phenyl-5H-dibenzo[b,d]thiophen-5-ium ((1S,4S)-7,7-dimethyl-2-oxobicyclo[2.2.1]heptan-1-yl)methanesulfonate in ethyl lactate; 0.198 gram of a 0.5 weight percent solution of fluorinated surfactant (Omnova PolyFox™ PF-656) in ethyl lactate; 12.411 grams of ethyl lactate; and 14.035 grams of 2-hydroxyisobutyric acid methyl ester. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

### Example 8

A positive-tone photoresist composition was prepared by combining 8.855 grams of a 10 weight percent solution of the Preparative Example 3 polymer in propylene glycol monomethyl ether acetate; 10.963 grams of a 2 weight percent solution of the acid generator 5-(4-(2-(1-methyladamantyl)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in propylene glycol monomethyl ether acetate; 0.531 gram of a 0.5 weight percent solution of tetrakis(2-hydroxypropyl)ethylenediamine in propylene glycol monomethyl ether acetate; 1.154 grams of a 2 weight percent solution of the Example 5 photo-destroyable quencher in propylene glycol monomethyl ether acetate; 0.098 gram of a 0.5 weight percent solution of fluorinated surfactant (Omnova PolyFox™ PF-656) in propylene glycol monomethyl ether acetate; and 18.410 grams of propylene glycol monomethyl ether acetate. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

### Comparative Example 6

A positive-tone photoresist composition was prepared by combining 17.712 grams of a 10 weight percent solution of the Preparative Example 3 polymer in propylene glycol monomethyl ether acetate; 21.941 grams of a 2 weight percent solution of the acid generator 5-(4-(2-(1-methyladamantyl)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in propylene glycol monomethyl ether acetate; 1.063 grams of a 0.5 solution of tetrakis(2-hydroxypropy)ethylenediamine in propylene glycol monomethyl ether acetate; 0.023 gram of the solvent-less Comparative Example 2 photo-destroyable quencher; 0.177 gram of a 0.5 weight percent solution of fluorinated surfactant (Omnova PolyFox™ PF-656) in propylene glycol monomethyl ether acetate; and 39.084 gram of propylene glycol monomethyl ether acetate. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

### Comparative Example 7

A positive-tone photoresist composition was prepared by combining 9.973 grams of a 10 weight percent solution of the Preparative Example 3 polymer in ethyl lactate; 11.650 grams of a 2 weight percent solution of the acid generator 5-(4-(2-(1-ethylcyclopentyloxy)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in ethyl lactate; 1.169 grams of a 0.5 weight percent solution of tetrakis(2-hydroxypropyl)ethylenediamine in ethyl lactate; 0.640 gram of a 2 weight percent solution of the photo-destroyable quencher 5-phenyl-5H-dibenzo[b,d]thiophen-5-ium ((1S,4S)-7,7-dimethyl-2-oxobicyclo[2.2.1]heptan-1-yl)methanesulfonate in ethyl lactate; 0.199 gram of a 0.5 weight percent solution of fluorinated surfactant (Omnova PolyFox™ PF-656) in ethyl lactate; 11.742 gram of ethyl lactate; and 14.625 grams of 2-hydroxyisobutyric acid methyl ester. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

### Comparative Example 8

A positive-tone photoresist composition was prepared by combining 7.952 grams of a 10 weight percent solution of the Preparative Example 3 polymer in ethyl lactate; 9.289 grams of a 2 weight percent solution of the acid generator 5-(4-(2-(1-ethylcyclopentyloxy)-2-oxoethoxy)-3,5-dimethylphenyl)-5H-dibenzo[b,d]thiophenium 3-hydroxyadamantane-acetoxy-1,1,2,2-tetrafluorobutane-1-sulfonate in ethyl lactate; 0.932 gram of a 0.5 weight percent solution of tetrakis(2-hydroxypropyl)ethylenediamine in ethyl lactate; 0.680 gram of a 2 weight percent solution of the photo-destroyable quencher 5-(4-(2-((1-ethylcyclopentyl)oxy)-2-oxoethoxy)-3,5-dimethylphenyl)-5H-dibenzo[b,d]thiophen-5-ium (1r,3s,5R,7S)-3-hydroxyadamantane-1-carboxylate in ethyl lactate; 0.159 gram of a 0.5 weight percent solution of fluorinated surfactant (Omnova PolyFox™ PF-656) in ethyl lactate; 9.287 grams of ethyl lactate; and 11.700 grams of 2-hydroxyisobutyric acid methyl ester. The resulting mixture was passed through a 0.01 micrometer polytetrafluoroethylene filter to yield the photoresist composition. The photoresist composition was spin coated onto a silicon wafer, soft baked to remove carrier solvent and exposed through a photomask to EUV radiation. The imaged resist layer was then baked at 100° C. for 60 seconds and then developed with an aqueous alkaline composition.

Shelf Life Testing.

Shelf life was determined by making a 100 millimolar solutions of triphenylsulfonium camphorsulfonate (structure shown below), triphenylsulfonium phenolate, and triphenylsulfonium pentafluorophenolate in deuterated dimethylsulfoxide. Compound stability was monitored by peak integration using ¹H NMR at 25° C. with relaxation delay set to 5 seconds on a 300 Megahertz Varian NMR spectrometer. Compound purity was calculated using the following formula: 100−((sum impurity peaks)/((total impurity peaks)+(total purity peaks))*100). The results, which are summarized in Table 1, show that triphenylsulfonium pentafluorophenolate is substantially more stable than triphenylsulfonium phenolate.

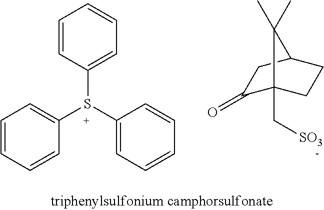

TABLE 1

| | Compound Purity (%) | | |
|---|---|---|---|
| Compound | 0 days | 3 days | 14 days |
| Triphenylsulfonium | 100 | 100 | 100 |
| camphorsulfonate | | | |
| (comparative) | | | |
| Triphenylsulfonium | 100 | 100 | 100 |
| pentafluorophenolate | | | |
| (inventive) | | | |
| Triphenylsulfonium | 100 | 79 | 60 |
| phenolate (comparative) | | | |

Water Absorption.

Compounds that are hygroscopic can pose a problem for formulation science and consequently lithographic results. Compounds that absorb atmospheric moisture may cause inaccurate amount additions into formulations and are also difficult to handle. The Comparative Example 1 compound (triphenylsulfonium phenolate) is a hygroscopic compound, as is the Example 2 compound (triphenylsulfonium pentafluorophenolate). However, it was observed that changing the cation from triphenylsulfonium to dibenzothiophenium reduced hygroscopicity. To quantify this characteristic, 1 millimole of each solid was placed in an open vial and enclosed in a 4 liter vessel containing 100 milliliters of exposed water. The weight of each vial was prerecorded and reweighed 18 hours later to determine the water weight gain. The results, summarized in Table 2, show that the inventive photo-destroyable quenchers of Examples 1-6 are less hygroscopic than the triphenylsulfonium phenolate of Comparative Example 1. The inventive photo-destroyable quenchers of Examples 2-5, each including an unsubstituted or substituted phenyl dibenzothiophenium ion, remained solid after exposure to water. The low hygroscopicity of the inventive compounds makes them easier to synthesize and isolate, and easier to handle and measure accurately during mixing of a photoresist composition.

TABLE 2

| | | Weight | Matter | Matter |
|---|---|---|---|---|
| | | Gain | State | State |
| | | relative to | Before | After |
| | | Reference | Exposure | Exposure |
| Compound | Cation Type | (%) | to Water | to Water |
| Triphenylsulfonium | Triphenylsulfonium | Reference | Solid | Solid |
| camphorsulfonate | | | | |
| (reference) | | | | |
| Compar. Example 1 | Triphenylsulfonium | 520% | Solid | Oil |
| Example 1 | Triphenylsulfonium | 29% | Solid | Sticky |
| | | | | Solid |
| Example 2 | Phenyl dibenzothiophenium | 43% | Solid | Solid |
| Example 3 | Phenyl dibenzothiophenium | 29% | Solid | Solid |
| Example 4 | Phenyl dibenzothiophenium | −14% | Solid | Solid |
| Example 5 | t-Butylphenyl | −29% | Solid | Solid |
| | dibenzothiophenium | | | |
| Example 6 | 3,5-Dimethyl-4-(((2- | −43% | Solid | Sticky |
| | methyladamantant-2-yl)oxy)-2- | | | Solid |
| | oxoethoxy)phenyl | | | |
| | dibenzothiophenium | | | |

Contrast. Table 3 presents the slope of the contrast curve using a CANON 248 nm exposure tool with a soft bake at 110° C. for 90 seconds, a post exposure bake for at 100° C. for 60 seconds, and development for 30 seconds at room temperature in 0.26 molar tetramethylammonium hydroxide developer. The contrast of Example 7 is normalized to 1, and designated with “⋄”. Comparative examples which underperform relative to the example by 0-5% are designated with“”; comparative examples which underperform relative to the example by 5%-15% are designated with “▪”; and comparative examples which underperform relative to the example by >15% are designated with “□”. High contrast correlates to good line fidelity at the mask edge which is related to good line width roughness (LWR) and critical dimension uniformity (CDU). Each of the photoresist compositions in Table 3 differ only in the identity of the quencher. The steepest contrast was observed for the Example 7 photoresist composition comprising the Example 5 quencher, 5-(4-(tert-butyl)phenyl)-5H-dibenzo[b,d]thiophen-5-ium 2,3,4,5,6-pentafluorophenolate. Table 3 also includes pKₐ values for the conjugate acid of the quencher anion. pKₐ values in aqueous solution at 23° C. were calculated using Advanced Chemistry Development (ACD) Labs Software Version 11.02.

TABLE 3

| | | | pKₐ of |
|---|---|---|---|
| | | | conjugate |
| | | | acid of |
| Photoresist | | Normalized | quencher |
| Composition | Quencher | Contrast | anion |
| Example 7 | 5-(4-(tert-Butyl)phenyl)-5H- | ⋄ | 5.50 |
| | dibenzo[b,d]thiophen-5-ium 2,3,4,5,6- | | |
| | pentafluorophenolate | | |
| Compar. | 5-(4-(tert-butyl)phenyl)-5H- | | 4.60 |
| Example 4 | dibenzo[b,d]thiophen-5-ium (1r,3s,5R,7S)-3- | | |
| | hydroxyadamantane-1-carboxylate | | |
| Compar. | 5-(4-(tert-Butyl)phenyl)-5H- | ▪ | −8.66 |
| Example 3 | dibenzo[b,d]thiophen-5-ium cyclohexylsulfamate | | |
| Compar. | 5-phenyl-5H-dibenzo[b,d]thiophen-5-ium | □ | 1.17 |
| Example 5 | ((1S,4S)-7,7-dimethyl-2- | | |
| | oxobicyclo[2.2.1]heptan-1-yl)methanesulfonate | | |

The Table 3 data were for photoresist compositions in which the primary solvent was ethyl lactate. Table 4 presents similar data for photoresist compositions in which the primary solvent was propylene glycol monomethyl ether acetate. Contrast values were normalized to Example 8. The steepest contrast was observed for the Example 8 photoresist composition comprising the Example 5 quencher, 5-(4-(tert-Butyl)phenyl)-5H-dibenzo[b,d]thiophen-5-ium 2,3,4,5,6-pentafluorophenolate.

TABLE 4

| | | | pKₐ of |
|---|---|---|---|
| | | | conjugate |
| | | | acid of |
| Photoresist | | Normalized | quencher |
| Composition | Quencher | Contrast | anion |
| Example 8 | 5-(4-(tert-Butyl)phenyl)-5H- | ⋄ | 5.50 |
| | dibenzo[b,d]thiophen-5-ium 2,3,4,5,6- | | |
| | pentafluorophenolate | | |
| Compar. | 5-(4-(tert-Butyl)phenyl)-5H- | | −8.66 |
| Example 6 | dibenzo[b,d]thiophen-5-ium cyclohexylsulfamate | | |

Critical Dimension Uniformity.

Critical dimension uniformity (CDU) is the calculated 3 Sigma (three standard deviations) for ten Fields of View (FOV) measuring 36 contact holes for each FOV, all taken at Best Exposure/Best Focus. Each data point has been pre-normalized to a standard EUV photoresist which is run in each lithographic slot to eliminate variability and noise. The results, presented in Table 5, show that the lowest (best) CDU value is exhibited by the inventive Example 7 photoresist with the inventive Example 5 photo-destroyable quencher 5-(4-(tert-butyl)phenyl)-5H-dibenzo[b,d]thiophen-5-ium 2,3,4,5,6-pentafluorophenolate.

TABLE 5

| | | | pKₐ of |
|---|---|---|---|
| | | | conjugate |
| | | CDU | acid of |
| Photoresist | | normalized | quencher |
| Composition | Quencher | to Example 7 | anion |
| Example 7 | 5-(4-(tert-Butyl)phenyl)-5H-dibenzo[b,d]thiophen- | 1 | 5.50 |
| | 5-ium 2,3,4,5,6-pentafluorophenolate | | |
| Comparative | 5-(4-(2-((1-ethylcyclopentyl)-oxy)-2-oxoethoxy)- | 1.04 | 4.60 |
| Example 8 | 3,5-dimethylphenyl)-5H-dibenzo[b,d]thiophen-5- | | |
| | ium (1r,3s,5R,7S)-3-hydroxyadamantane-1- | | |
| | carboxylate | | |
| Comparative | 5-Phenyl-5H-dibenzo[b,d]thiophen-5-ium ((1S,4S)- | 1.30 | 1.17 |
| Example 7 | 7,7-dimethyl-2-oxobicyclo[2.2.1]heptan-1- | | |
| | yl)methanesulfonate | | |
| Comparative | 5-(4-(tert-Butyl)phenyl)-5H-dibenzo[b,d]-thiophen- | 1.04 | 4.60 |
| Example 4 | 5-ium (1r,3s,5R,7S)-3-hydroxyadamantane-1- | | |
| | carboxylate | | |
| Comparative | 5-phenyl-5H-dibenzo[b,d]thiophen-5-ium ((1S,4S)- | 1.07 | 1.17 |
| Example 5 | 7,7-dimethyl-2-oxobicyclo[2.2.1]heptan-1- | | |
| | yl)methanesulfonate | | |

1-4. (canceled)

5. A photo-destroyable quencher having the structure

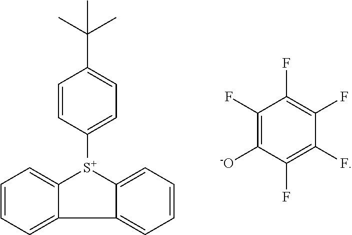

6-8. (canceled)

9. A photoresist composition comprising:
an acid-sensitive polymer;
a photoacid generator; and
a photo-destroyable quencher having the structure

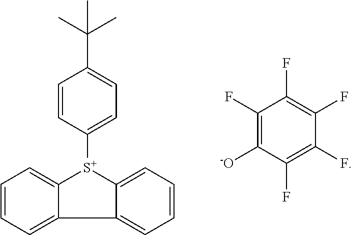

10. A method of forming an electronic device, comprising:
(a) applying a layer of a photoresist composition of claim 9 on a substrate;
(b) pattern-wise exposing the photoresist composition layer to activating radiation; and
(c) developing the exposed photoresist composition layer to provide a resist relief image.
